(19) United States
(12) Patent Application Publication
(10) Pub. No.: US 2006/0046256 A1
(43) Pub. Date: Mar. 2, 2006

(54) Identification of informative genetic markers

(75) Inventors: Bjarni V. Halldorsson, Reykjavik, IC (US); Vineet Bafna, San Diego, CA (US); Ross A. Lippert, Cambridge, MA (US); Russell Steven Schwartz, Pittsburgh, PA (US); Francisco De La Vega, San Mateo, CA (US); Andrew Clark, Ithaca, NY (US); Sorin Istrail, Rockville, MD (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/040,652

(22) Filed: Jan. 21, 2005

Related U.S. Application Data
(63) Continuation of application No. 60/537,619, filed on Jan. 20, 2004.

(51) Int. Cl. C12Q 1/68; G06F 19/00

(52) U.S. Cl. 435/6; 702/20

(57) ABSTRACT

The present teachings describe methods for selecting informative genetic markers including single nucleotide polymorphisms (SNPs) that may be used in the design and execution of genome wide association studies. These methods are distinguished from other methods relying on a predefined haplotype block structure and may be configured to make use of correlations that occur across neighboring haplotype blocks. The disclosed methods may further be implemented across chromosomal regions having both high and low local linkage disequilibrium. Informative genetic marker selection, as described, provides an alternative and potentially more efficient mechanism to select genetic markers such as SNPs using block-based and random approaches.

### CROSS-REFERENCE TO RELATED APPLICATIONS

This application claims the benefit of U.S. Provisional Application No. 60/537,619, entitled “Optimal Block-Free Selection of Tagging SNPs”, filed on Jan. 20, 2004, which is hereby incorporated in its entirety by reference.

### FIELD

The present teachings generally relate to the field of genetic analysis and more particularly to methods for selection of single nucleotide polymorphisms.

### BACKGROUND

Single nucleotide polymorphisms (SNPs) are one of the most abundant forms of genetic variation in biological organisms. It has been suggested that single nucleotide changes occur with an approximate frequency of one in every 500 base pairs in the human genome. Analysis of SNPs may be conducted at various levels including genome-wide, group-wise, and individually. Each of these approaches may prove useful in a variety of biological applications and pharmacological studies.

One problem that arises during SNP analysis is how to reduce the quantity information to be studied. For example, evaluating large numbers of SNPs across a genome may present issues of computational and analytical complexity. In certain instances, it may be desirable to reduce or minimize the number of SNPs being studied and isolate groupings or subsets of SNPs relevant, informative, and/or representative of selected conditions and/or haplotypes of interest. In such SNP groupings, it is generally desirable to retain characteristics of informativeness that may be reflected by a larger subset of SNPs while eliminating redundancies and less-informative SNPs. In attempts to define specific relationships between SNPs and various disease states it is also desirable to identify minimal SNP subsets or groupings of significance.

Various methods for selecting and investigating SNP subsets have been proposed. One approach involves evaluating contiguous SNP segments along one or more chromosomes to identify haplotype blocks. A haplotype block represents a contiguous series of SNPs exhibiting low haplotype diversity across individuals and with generally low recombination frequencies. Within a given haplotype block, SNPs may be highly correlated and the genotypes of one SNP can be used to predict the genotypes of another. Such block-based methods for analysis have various drawbacks however, including a general lack of a consensus as to how to best define haplotype blocks for a selected region. Furthermore, determining the subset of SNPs that provides the most useful discriminatory or informative potential within a selected haplotype block can be problematic.

Another problem is that there are multiple competing methods that generate different haplotype block structures within the scientific community and a lack of agreement on metrics to quantify block quality. Additionally, substantial correlation may exist between sets of adjacent haplotype blocks, implying that there may be useful information that traverses conventionally rigid block boundaries. Consequently there is a need for improved methods by which to identify correlations between SNPs and SNP subsets which are not limited by conventional haplotype block identifications. Furthermore, there is a need for improved mechanisms by which to identify subsets of SNPs capable of reflecting similar information as larger SNP groups.

### SUMMARY

In various embodiments the present teachings describe methods for identifying or selecting informative sets or groupings of genetic markers. According to certain embodiments of the present teachings selection of an informative genetic marker set may be accomplished through processes in which genetic markers under consideration are partitioned according to the steps of: (a) Determining neighborhoods or localities wherein tagging genetic markers are evaluated to identify candidate sets of genetic markers that can be meaningfully used to infer each other; (b) Performing tagging quality assessments for the selected genetic markers that may be used to characterize how a genetic marker set captures observed/expected variances; and (c) Performing genetic marker set optimization operations to reduce or minimize the number of genetic markers within the set.

In another aspect, the present teachings describe a method for analyzing nucleotide sequence information. This method comprises: selecting a data collection comprising information describing a plurality of genetic markers; determining informativeness for selected genetic markers within the data collection based at least in part upon an identification of at least one neighborhood wherein allelic states for two or more genetic markers of the data collection are correlated and wherein the informativeness for a selected genetic marker is reflected by the predictive value of the selected genetic marker in identifying allelic states for other genetic markers within the region; and evaluating the informativeness of the genetic markers within the data collection to identify a genetic marker subset that possesses a selected degree of predictive quality in predicting other genetic markers.

In still another aspect, the present teachings describe a method for analyzing nucleotide sequence information wherein the method comprises: selecting a data collection comprising genetic marker information describing a plurality of genetic markers; determining at least one neighborhood comprising a plurality of genetic markers associated with the data collection; identifying genetic markers associated with the at least one neighborhood having correlated allelic states; determining at least one set of genetic markers selected from those genetic markers associated with the at least one neighborhood that infer one another; and associating with the at least one set of genetic markers, a quality measure that reflects how well the one set of genetic markers can be used to characterize other genetic markers of the data collection.

In a further aspect, the present teachings describe a system for analyzing nucleotide sequence information. This system comprises: a data collection component that provides functionality for selecting a data collection comprising information describing a plurality of genetic markers; a computational component that provides functionality for determining informativeness for selected genetic markers within the data collection based at least in part upon an identification of at least one region wherein allelic states for two or more genetic markers of the data collection are correlated and wherein the informativeness for a selected genetic marker is reflected by the predictive value of the selected genetic marker in identifying allelic states for other genetic markers within the region; and a data analysis component that provides functionality for evaluating the informativeness of the genetic markers within the data collection to identify a genetic marker subset that has a selected degree of predictive quality in predicting other genetic markers.

In a still further aspect, the present teachings describe an apparatus comprising a computer readable medium having instructions stored thereon to analyze nucleotide sequence information by the steps of: selecting a data collection comprising genetic marker information describing a plurality of genetic markers; determining at least one neighborhood associated with the data collection; identifying genetic markers associated with the at least one neighborhood; determining at least one set of genetic markers selected from those genetic markers associated with the at least one neighborhood that infer one another; and associating with the at least one at least one set of genetic markers, a quality measure that reflects how well the one set of genetic markers can be used to characterize other genetic markers.

### BRIEF DESCRIPTION OF THE DRAWINGS

The present invention will become more fully understood from the detailed description and the accompanying drawings, wherein:

FIG. 1 illustrates an overview of a method for identifying exemplary genetic markers comprising tagging SNPs and selection of an informative SNP set.

FIG. 2 illustrates details of a method for selection of exemplary genetic markers comprising tagging SNPs and SNP subsets.

FIG. 3 illustrates a neighborhood graph used to reflect the predictability of exemplary genetic markers comprising SNPs in relation to one another.

FIG. 4 illustrates a method for refining an informative SNP set of genetic markers.

FIG. 5 illustrates pseudocode for a method that may be adapted for computation of an informative genetic marker set of SNPs.

FIG. 6 illustrates a block diagram of a system for conducting analysis of exemplary genetic marker SNPs according to the present teachings.

FIG. 7 illustrates a table for imputing haplotypes as described by the present teachings.

FIG. 8 is a graph showing how the definition of informativeness described by the present teachings correlates with a practical benchmark of the value of an SNP subset.

### DETAILED DESCRIPTION

As used herein, unless otherwise explicitly stated, the terms “a,” “an,” “the,” “said,” and “at least one” are not intended to be limited in number to “one,” but rather are intended to be read as encompassing “more than one” (i.e., a plurality) as well. Also, “a probe” is intended to include two bi-allelic probes in the case of SNP assays, unless stated otherwise. Where examples are recited herein, such examples are intended to be non-limiting.

In various embodiments the present teachings describe methods for identifying or selecting sets of genetic markers (e.g. for example, single nucleotide polymorphisms (SNPs), microsatellites, insertions, deletions, biallelic polymorphisms, and multiple nucleotide polymorphisms) associated with genetic regions of interest. These genetic marker sets may be used for numerous purposes, including for example, providing selecting sequence indicators associated with various conditions and locating genetic markers that may be associated with traits of biological and/or pharmacological relevance. As used herein the informativeness of a genetic marker set may refer to the efficiency or utility with which the genetic marker set may be used to characterize one or more desired characteristics, traits, classes, phenotypic pools, haplotypes, or other information. Informativeness of the genetic marker set may also be related to discriminating between selected individuals, samples, and/or populations. For purposes of illustrating and describing the approaches to genetic marker selection and analysis of the present teachings, single nucleotide polymorphisms serve as a genetic marker of interest. It will be appreciated however, that the present teachings may be extended to other types of genetic markers such as those indicated above and consequently it is conceived that the methods and approaches described herein are extensible to a plurality of different genetic marker types and compositions.

By way of introduction a gene, genetic region of a chromosome, or selected genetic/sequence loci may be associated with one or more. Genotyping of this region refers to the identification of the various allelic combinations of SNPs (e.g. sequence variants) and there occurrence within a selected sample. When determining a representative or candidate set of SNPs used to evaluate a genetic region/loci it may be desirable to reduce or minimize the number of SNPs within the set while at the same time preserving or maximizing the amount of discriminatory potential of the SNP set. Thus for an exemplary loci containing a designated number of SNPs, certain SNPs within the loci may provide redundant or duplicative information as other compared with SNPs within the loci. Thus, it may be the case that a smaller subset of SNPs may be able to represent a portion, substantially all, or all of the information provided by the large superset from which it was obtained.

According to various embodiments of the present teachings, an informative set or subset of SNPs reflects a grouping of SNPs that may be used to represent at least a portion of the allelic diversity within a selected locus. In general, an informative SNP set may be optimized in such a manner so as to reduce the number of SNPs within the set while preserving the informational content as described above. A minimal informative SNP set refers to a grouping of SNPs that preserves discriminatory potential (for example, within a selected percentage or threshold) while at the same time contains a reduced or minimum number of SNPs. In certain embodiments, application of the methods described herein provides mechanisms for selecting informative SNP sets (minimal and otherwise) and assessing the quality and/or effectiveness of the informative SNP sets for capturing/representing haplotype diversity. It may also be desirable to identify reduced informative SNP sets or minimal informative SNP sets, for example, to reduce analytical complexity associated with studying genetic loci and to improve computational throughput when implementing computer-based methods for SNP searching and analysis.

According to various embodiments, SNP set identification may include an analysis of linkage disequilibrium (LD) neighborhoods to restrict and/or focus attention to SNP trends or characteristics represented within a selected population. Linkage disequilibrium may refer to observed differences in frequencies of haplotypes or genetic markers (e.g. SNP combinations/sequences) within a sample or population. In one aspect, LD reflects a characteristic wherein the observed frequency of haplotypes does not agree with predicted or expected haplotype frequencies if individual SNPs or genetic markers within a selected haplotype are considered individually.

Conventional approaches to SNP selection for genome-wide analysis frequently focus on the problem of defining a quality measure. In general, such a quality measure may reflect an evaluation or characterization of how well a plurality of SNPs capture observed variances between samples, groups, and/or populations. Such assessments often require or infer the assumption that an analysis region is not very large and thus the number of candidate SNPs is not very large. Based on this, it may be assumed that most observed correlations are true correlations which may be incorrect in numerous instances.

In various embodiments, the methods described herein are an advancement over conventional methods in that they provide a mechanism by which to minimize or reduce the number of SNPs required to evaluate large data sets. Applying this approach to conventional methods for determining quality measures desirably improves the efficiency of evaluating even large numbers of SNPs. As will be described in greater detail herein below, evaluation of SNPs and SNP sets according to the present teachings may allow the selection of tagging SNPs that result in significant savings over selecting SNPs through conventional methods such as by random selection or haplotype block based approaches. In one aspect, tagging SNPs or haplotype tagging SNPs refer to SNPs identified according to the described methods that may be used to characterize genetic diversity within a selected region or genetic sequence. Groups of tagging SNPs may further be used to form a SNP subset that allows reconstruction, prediction, or identification of a group of one or more SNP genotypes.

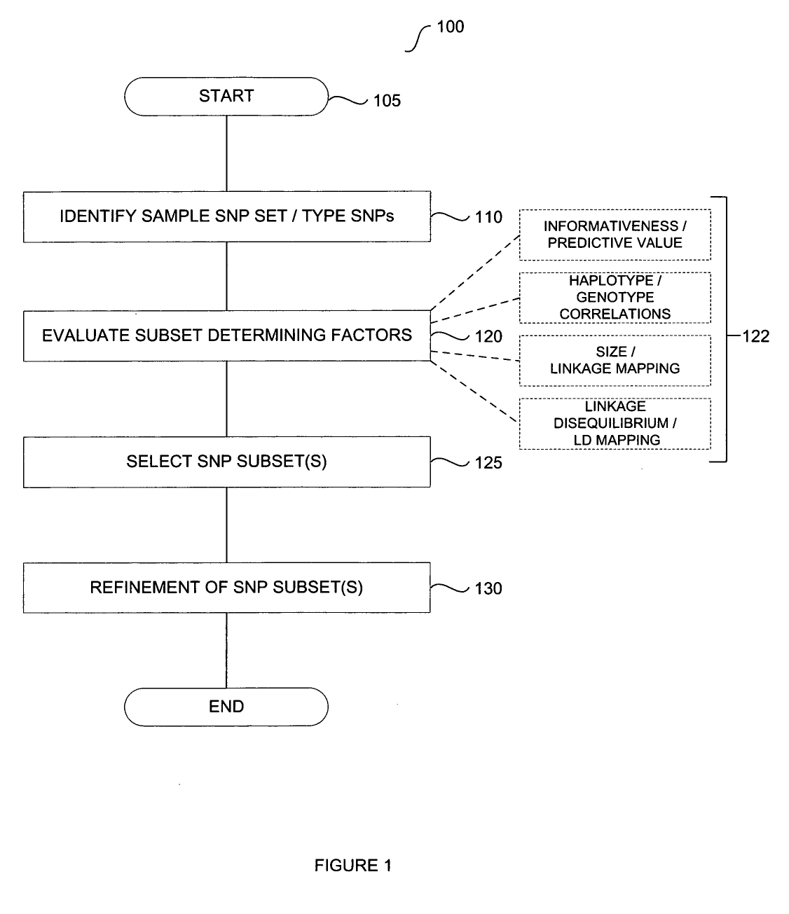

FIG. 1 illustrates an overview of a method 100 for identifying tagging SNPs and selection of an informative SNP set. This method may be used in connection with a plurality of candidate SNPs which are partitioned, grouped, or categorized to identify an informative SNP subset. The informative SNP subset may provide a desired degree of discriminatory potential for evaluating a selected genetic locus while at the same time reducing to number of SNPs required (e.g. increasing informativeness).

In various embodiments, the approach to selection of the informative SNP subset proceeds on the basis of haplotype tagging wherein subsets of SNPs are identified so as to represent common haplotypes inferred from an original SNP set or collection. Tagging or Tag SNPs further relate to SNPs having a characteristic of informativeness in which a selected SNP may be used to report partially or totally a state associated with other SNPs (e.g. sequence/genotype/haplotype/allelic composition). In one aspect, these other SNPs may not be necessary to genotype because their state is reported by one or a combination of tagging SNPs. Consequently, the amount of SNP typing or information required to represent the state for a selected genetic loci or collection of SNPs may be reduced significantly.

In one exemplary application, genetic association studies may be facilitated by the informative SNP subset selection method. For example, a region (e.g. up to a full chromosome) or a set of regions (e.g. candidate genes) of interest may be identified as an area of interest in which an investigator may desire to conduct a search for sequence variants associated with a selected phenotypic trait. A defined study population may further be selected and a preliminary survey of substantially all or a subset of all SNPs in the region(s) may be performed on a representative sample from the population (e.g. one or more individuals). This type of data may further arise for example from resequencing a subset of the individuals or from a large genotyping studies in reference samples such as the HapMap project.

In this context, the investigator may desire to identify subsets of SNPs that allow reconstruction of some, all, or substantially all of the observed or present sequence variation present within the population. In a general sense, this scenario bears some degree of similarity to a data compression problem: The input may be considered as a full pattern of available or surveyed SNPs for a relatively small sample and the goal may be to devise an approach to select SNPs that will allow the reconstruction of the full data set. In this situation, it may be desirable that while only a subset of all SNPs are genotyped, the statistical power for identifying phenotype-genotype associations would be minimally compromised, or that its decrease can be compensated by a reasonable increment in the study's sample size.

As in the example described above and in certain embodiments, the SNP subset provides the potential to reconstruct a portion, substantially all, or all of the haplotypes inferred by genotyping other previously know SNPs associated with a selected locus or larger scale or region (e.g. genome-wide). The method for SNP selection of the present teachings may also provide the ability to select from available SNPs for a sample, region, or loci and reconstruct a portion, substantially all or all of the data (e.g. haplotypes/allelic variants) associated with a larger SNP data set. One application of this principle may be to identify DNA sequence variation within a selected population associated with a selected trait or condition (for example, an LD-associated, elevated risk of disease, adverse drug reaction, or drug efficacy analysis).

Referring again to FIG. 1, the method 100 proceeds from a start state 105 to a state 110 wherein a sample/candidate set of SNPs is identified. These SNPs may be typed for the analysis or obtained from a database of available SNP information. The SNP sample set represents available SNPs that may be selected from and used in the identification of useful SNP subsets determined by evaluating desirable factors and characteristics. In state 120, an evaluation of these subset determining factors is performed across at least a portion of the SNP sample set. There are a number of factors 122 that may be used in this determination which may include consideration of: informativeness size/predictive value of the SNPs; haplotype/genotype correlations; size/linkage mapping characteristics; and or linkage disequilibrium/LD mapping characteristics. In various embodiments and as will be described in greater detail herein below, these characteristics are used in the determination of the potential for selected candidate SNPs to represent information provided by other SNPs. Thus the selected candidate SNPs allow a SNP subset to be devised which possesses a selected degree of discriminatory potential that may approach that of the data superset from which it was derived.

In state 125, one or more SNP subsets are identified using information obtained in step 120 above wherein SNP subsets having characteristics that may be useful in subsequent analysis or can be used to represent portions of a selected genetic region or loci. In state 130, refinement of the one or more SNP subsets may take place to achieve further optimized or minimized SNP subsets.

As will be described in greater detail hereinbelow, the one or more SNP subsets identified by application of this method 100 may be customized according to various parameters, thresholds, and data used. In general, the resulting SNP subsets may desirably reflect haplotype tagging SNPs that may be used to characterize the overall genetic diversity of a selected genetic region or loci. The tagging SNPs that make up the subset may further provide sufficient information to reconstruct at least a portion of the full set of haplotypes determined by the entirety of the sample set identified in state 110. In doing so, selected tolerances for correctness or completeness in determination of the complete set of haplotypes may be considered when determining the composition of the tagging SNPs within the SNP subset.

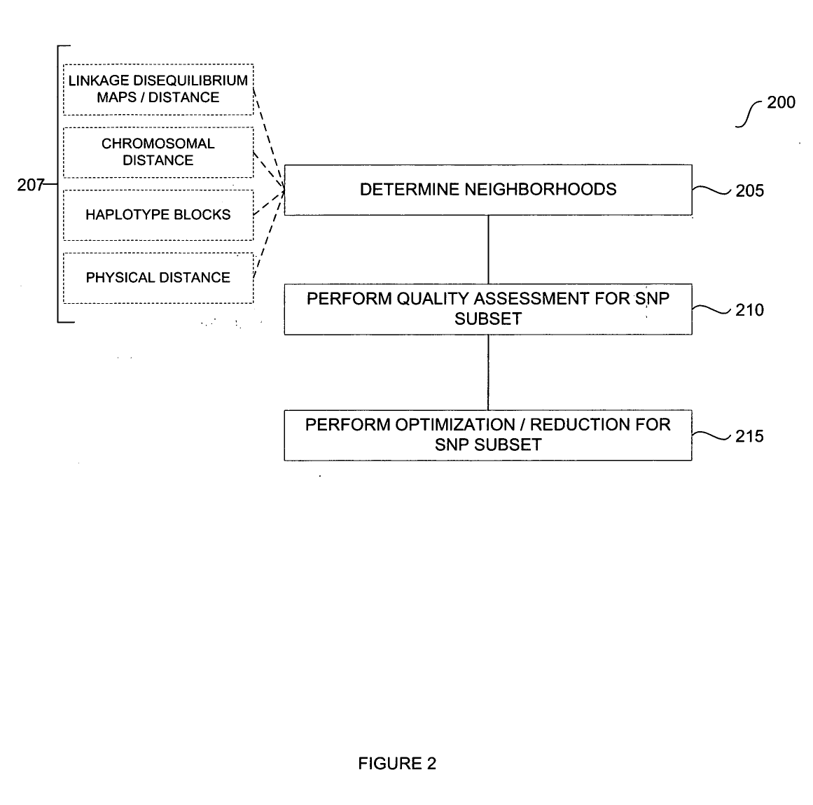

The selection of tagging SNPs and SNP subsets as described above is further illustrated in FIG. 2. In various embodiments, a method 200 comprising the indicated operations may be used to select the tagging SNPs. These operations include determining predictive neighborhoods comprising pluralities of SNPs that can be used to infer each other as illustrated by step 205; performing a tagging quality assessment for selected SNP subsets as illustrated by step 210 wherein quality measures are defined and used to characterize how well a SNP subset captures observed/expected variances in the data superset from which it was derived, and performing an optimization or reduction of the selected SNP subsets as illustrated by step 215 to reduce the number of tagging SNPs within the selected SNP subsets while maintaining a desired quality threshold. It will be appreciated that each of the aforementioned steps may be operated largely independently of the others. Thus methods for performing one of these steps 205, 210, 215 may be devised and combined with other methods for performing another of the steps 205, 210, 215.

As will be described in greater detail hereinbelow the above-indicated operations may be associated with performing pairwise evaluations of SNPs to assess the ability of SNP pair members to be used to infer each other. Such an analysis may be useful when dealing with large regions, wherein occasional, spurious, and/or long-range correlations can be observed that may not necessarily be biologically relevant. SNP analysis in the aforementioned manner may also prove useful in overcoming a problem observed when dealing with very large numbers of SNPs, for example on a on a genome-wide scale and/or across populations, wherein a pair of SNPs may be found to be correlated by random chance and not necessarily extensible across an overall population.

The determination of predictive neighborhoods in state 205 may utilize a number of different approaches including by way of example: linkage disequilibrium maps/distance, chromosomal distance, haplotype blocks, and physical distance 207. In general, predictive neighborhood selection comprises identifying a selected interval where tagging SNPs are determined which are capable of inferring or reporting the state of other SNPs. When selecting tagging SNPs, it may be desirable to select without requisite a priori genotype information such as based on chromosomal distance. Such an approach may attempt to select one or more representative SNPs for each region of the genome.

Additionally, haplotype blocks may be used in the determination of neighborhoods. Briefly, haplotype block determination may proceed by partitioning a chromosome or genetic region of interest into contiguous segments and select, within each segment, a subset of “haplotype tagging” SNPs sufficient to reconstruct the haplotype diversity within the block.

A further approach to neighborhood determination may include as a measure, distances on linkage disequilibrium maps or physical distances. In various embodiments, using linkage disequilibrium assessments to identify LD neighborhoods provide useful mechanisms to focus on extensible trends and allow for improved analytical capabilities.

A significant advantage of the present teachings over conventional approaches to SNP identification is that the selection and use of neighborhoods of predictive SNPs provides a more flexible framework for SNP analysis. Furthermore, the present teachings describe metrics or measures useful for not only assessing informativeness of a selected SNP that may be related to measures of haplotype diversity, but also provides a more direct measure of how a given SNP, or a set of SNPs, may be used to characterize another SNP, or a set of SNPs. In one aspect, this assessment may be determined according to a computable function which is shown to correlate with other alternative conventional multiloci linkage disequilibrium measures. While the computation of sets of SNPs with minimal informativeness may be characterized as NP-hard in the general case, the present teachings demonstration that SNP set solutions may be tractable in many cases of practical interest especially when the number of SNPs predicting each SNP is small. The present teachings further provide methods for obtaining reduced or minimal sets of tagging SNPs in a computationally efficient or tractable manner.

In one exemplary approach to finding neighborhoods of potentially predictive SNPs, significant LD may be observed to occur between SNPs physically distant or separated within the genome. Such distant relationships may reflect common ancestry in the case of very recent admixture. Conventionally, if high LD is observed at greater distances than a selected threshold, such as 200 kb, it is commonly ignored and may be considered to be artifactual in nature. According to various embodiments of the present teachings identifying SNPs in LD with one another as illustrated in step 205 in FIG. 2 provides a mechanism to identify sets or groupings of SNPs that may be predictive not only of other SNPs in a selected sample, but also for those SNPs that may not have typed or been identified within a selected sample population. Thus, in performing this operation 205 it may be desirable to identify those SNPs that characterize regions of common recent ancestry (conserved haplotypes) rather than those that characterize isolated distant SNPs due to selection bias or random chance. One rationale for this may be that SNPs of recent common ancestry may be more likely to be those that are sufficiently close that recombination events have not occurred frequently between them. Consequently, it may be advantageous to search for predictive or informative SNPs that are in relatively close proximity to the targets for which they may be predictive.

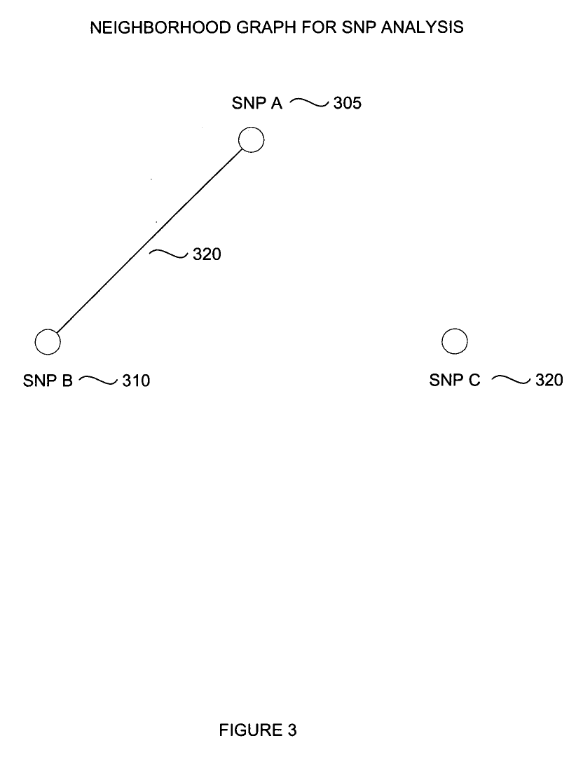

It is observed that recombination rates and historical LD vary across the genome. Additionally, variabilities in SNP density and recombination rate result in observed variability in SNP density and recombination rate for most SNPs. According to the present teachings, such variabilities may provide useful information. For example, as shown in FIG. 3 a neighborhood graph 300 may be constructed to reflect the predictability of SNPs in relation to one another. In such a graph 300, SNPs 305, 310, 315 may be represented as vertices. SNPs 305, 310 that are useful in predicting one another may be connected by an edge 320. Those SNPs 305, 320 and 310, 320 that are not useful in predicting one another may remain unconnected. Represented mathematically for each SNP s a set of neighbors may be defined as N(s).

In determining the utility in one SNP for predicting another, a determination may be made that assesses whether the allelic states of the two SNPs are correlated. Various statistics may be devised for quantifying this correlation (e.g. linkage disequilibrium) and determining if one SNP may be useful in predicting another even with little evidence of recombination between the two SNPs. In various embodiments, for a given method effective in detecting regions of low recombination rate and recognition that low recombination rate between SNPs potentially allows one SNP to predict another, then the set of SNPs that can be used to predict a selected SNP may be found by the union of decompositions by a block-free method as described in greater detail hereinbelow. Consequently, application of broader LD correlations potentially allow characterization of informational content within a selected region or loci with fewer SNPs, each of which has predictive power.

In consideration as to how to define informativeness 122 of a selected SNP or subset of SNPs it may be useful to define a measure of how well a subset of SNPs can predict a single target SNP. According to the present teachings, informativeness 122 reflects a measurement or assessment of how well one can we reconstruct a target SNP, t, from a set of its neighbors, N(t). In one aspect, given the haplotype pattern of set of its neighbors, one may look at the pairs of haplotypes that have a different allele at t, and count or assess how many of these also do not have the same set of alleles for the SNPs in N(t) and thereafter divide by the total number of pairs. This approach represents an example of a possible measure useful in defining informativeness 122.

Further evaluating informativeness 122, a set S of n SNPs over a population of m haplotypes may be denoted by an n×m matrix M. In such a matrix, the columns of M may reflect a correspondence to SNPs within a selected population (e.g. the sample SNP set 110), and the rows of M may correspond to haplotypes. Furthermore, for a SNP s, sᵢ may be denoted as M[i, s]. With the assumption that SNPs are biallelic (taking on only two values) for simplicity;
M[i, s]ε{0, 1}∀i, s Equation (1)

On this basis, a target SNP t may be associated with a trait of interest wherein if t is not typed, its state may be predicted using proximal SNPs. In various embodiments, a measure of informativeness 122 of a SNP s with respect to t may be used to quantify the accuracy with which such a prediction can be made. For a SNP s and haplotypes i, j, D^{s}_{i,j} reflects an event that M[i,s]≠5 M[j, s]. Thus the informativeness 122 of SNP s with respect to a SNP t may be characterized as;
I(s, t)=Prob_{i≠j}(D_{i,j}^{s}|D_{i,j}^{t}) Equation (2)

In the above equation, i, j represent two haplotypes drawn substantially uniformly at random from the set of all substantially distinct haplotype pairs. It may be observed that as I(s, t) approaches 1 substantially complete predictability is implied, and as I (s, t) approaches 0 substantially no predictability is implied. Consequently, I(s, t) may be readily estimated from a sample as follows:

Consider the complete graph G_{H} on m nodes labeled 1, 2, . . . , m. Each SNP s may be construed as representative of a subgraph which is a bipartite clique with m nodes. Each edge set E(s) may further be defined by a rule (i, j) [symbol] E(s) if and only if Sᵢ Sⱼ. Consequently the informativeness of s with respect to t may be reflected in the equation;

$$I ( s , t ) ≃ \frac{\vert E ( s ) ⋂ E ( t ) \vert}{\vert E ( t ) \vert} \qquad Equation ( 3 )$$

Such a definition may further be extended to a subset of SNPs wherein for S′ ⊂ S, D^{S′}_{i,j} reflects the event that M[i,s]≠M[j,s] for some s [symbol] S′. Likewise, (S′)=∪_{sS′}E(s). Therefore:

$$I ( {S}^{′} , t ) = {Prob}_{i \neq j} ( {D}_{i , j}^{{S}^{′}} \vert {D}_{i , j}^{t} ) ≃ \frac{\vert E ( {S}^{′} ) ⋂ E ( t ) \vert}{\vert E ( t ) \vert} \qquad Equation ( 4 )$$

When predicting a set of SNPs from a subset of tagging SNPs 125 a SNP is predicted from its set of neighbors and for S′, T ⊂ S resulting in:

$$I ( {S}^{′} , T ) = {\sum }_{t ∈ T} I ( {S}^{′} ⋂ N ( t ) , t ) \qquad Equation ( 5 )$$

On the basis of the aforementioned definition of informativeness 122 availability of haplotype phases may be expected. In practice, genotype data may be more easily determined experimentally than haplotypes, however, computationally inferring haplotypes over each neighborhood may be accomplished using a maximum likelihood/expectation maximization approach.

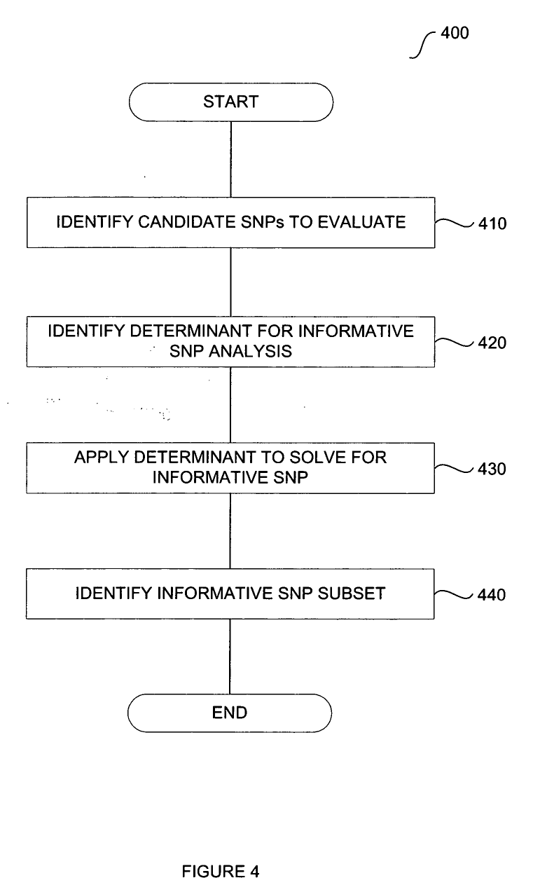

FIG. 4 illustrates a method 400 for optimizing or improving a measure of informativeness 122 as previously illustrated in FIG. 1. This method 400 may operate upon metrics such as those presented above or configured to operate in connection with other conventional metrics. In one aspect, the method 400 commences in a start state 405 and proceeds to a state 410 wherein one or more candidate SNPs are identified for use in evaluating informativeness. In one aspect, informativeness for a selected SNP is defined by a metric in state 420 and subsequently evaluated in the context of solving for a k-most informative SNP in state 430 to identify informative SNPs to be selected for the SNP subset in state 440 wherein:

k-MIS may reflect the k most informative SNPs with input received as a set of n SNPs S (e.g. from step 410) with 0<k<n and output determined by finding the subset S′ ⊂ S such that I(S′, S)=max_{R⊂S,|R|≦kl}(R, S).

Considering again the informativeness of a subset S′ with respect to a SNP t. The distance between SNPs s and t may be defined as the number of SNPs in between s and t. The minimum informative SNPs problem can be solved with the understanding that neighborhoods are not overly large (e.g. their size is bounded by some constant, for example 13 or 21). In one aspect, it is desirable to identify a principle informative subset of SNPs provided that SNPs that are a distance w apart can be used in the prediction. The k most informative SNPs, k-MIS, can be solved when the size of each neighborhood is bounded by a constant w. When enumerating the n SNPs from 1 to n it may be supposed that substantially all SNPs within distance └w/2∃ of s may be used to predict s. From this, the corresponding assignment Aₛ may be defined as follows:

$${A}_{s} [ i ] = \{ \begin{matrix}1 & if SNP s - ⌊ \frac{w}{2} ⌋ + i ∈ {S}^{′} \\ 0 & otherwise\end{matrix} \qquad Equation ( 6 )$$

Correspondingly, the subset of SNPs S(Aₛ) may be defined to contain a portion, substantially all, or all SNPs s′ such that

$${A}_{s} [ {s}^{′} + ⌊ \frac{w}{2} ⌋ - \vert s ] = 1 \vert \qquad Equation ( 7 )$$

If can further be demonstrated that the k-MIS problem can be solved O(nk2^{w}) time, and O(k2^{w}) space, if the size of all neighborhoods is bounded by a constant w. Here a solution to the k-MIS problem can be described by a 0(n) size bit-vector, such that B[i]=1 if SNP i is selected, and 0 otherwise. In general, at most k entries are 1 in any solution. The solution also implies an assignment As, for each SNP s as:

$${A}_{s} [ i ] = B [ s - ⌊ \frac{w}{2} ⌋ + i ] \qquad Equation ( 8 )$$

Letting A^{o}ₛ and Aₛ represent vectors obtained by removing the rightmost element of Aₛ and moving the other elements by one to the right and adding a 0 or 1 as the leftmost element. In any solution:

$$\begin{matrix}{A}_{s} [ i ] = B [ s - ⌊ \frac{w}{2} ⌋ + i ] \\ = B [ ( s - 1 ) - ⌊ \frac{w}{2} ⌋ + ( i + 1 ) ] \\ = {A}_{s - 1} [ i + 1 ]\end{matrix} \qquad Equation ( 9 )$$

Therefore, depending on whether the Aₛ₋₁[0] is 0 or 1, Aₛ₋=A⁰ₛ, or Aₛ₋₁=A¹s. Letting I_{w}(s,1, Aₛ) be the score of principle informative subset of 1 SNPs chosen from SNPs 1 through s, such that Aₛ described the assignment for SNP s. The score obtained for informing SNP s is I(S(Aₛ), s), and I_{w}(s, 1, Aₛ) is reflected by I(S(Aₛ), s) plus score of the best assignment for SNPs 1 through s-1 that is consistent with Aₛ.

It will be appreciated that from the foregoing description above, there are two possibilities for the assignment to SNP s-1, described by A⁰ₛ, A¹ₛ₋₁. Finally, the assignment to SNPs 1 through s-1 does not use SNP

$$s + ⌊ \frac{w}{2} ⌋ \vert .$$

Therefore, the number of SNPs available to SNPs 1 through s-1 are I-1 if Aₛ[w]=1, and I otherwise.

Thus
I_{w}(s,l,Aₛ)=I(S(Aₛ),s)+max(I_{w}(s-1, l-Aₛ[w], Aₛ⁰), I_{w}(s-1,l-Aₛ[w],Aₛ¹) Equation (10)

In one aspect, the score of the optimal assignment for choosing k SNPs from the whole set can be retrieved as max_{An} 1_{w}(s, k, Aₙ) and the optimal assignment can be retrieved via a backward traversal. This approach may further be reduced to the space requirements of O(k2^{w}). Also, as many if not most neighborhoods will be smaller than the maximum size w, efficiency gains can be made in implementation.

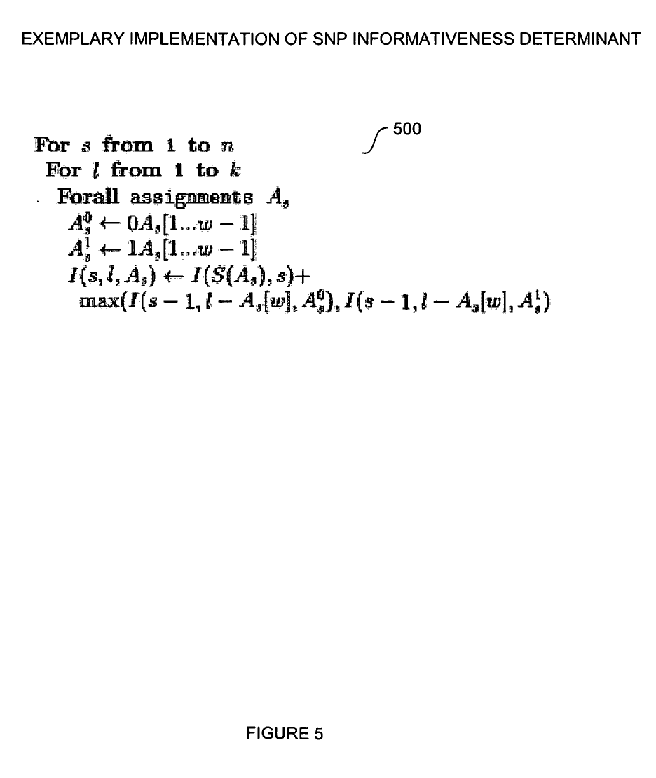

FIG. 5 illustrates pseudocode 500 corresponding to a method that may be adapted for computation of this recurrence using dynamic programming. As described above, this approach may be configured to assume a maximum size of w on each neighborhood and may be computed in O(nk2^{w}). A source code implementation of the aforementioned method 500 may be implemented in a computerized system for SNP analysis according to the present teachings.

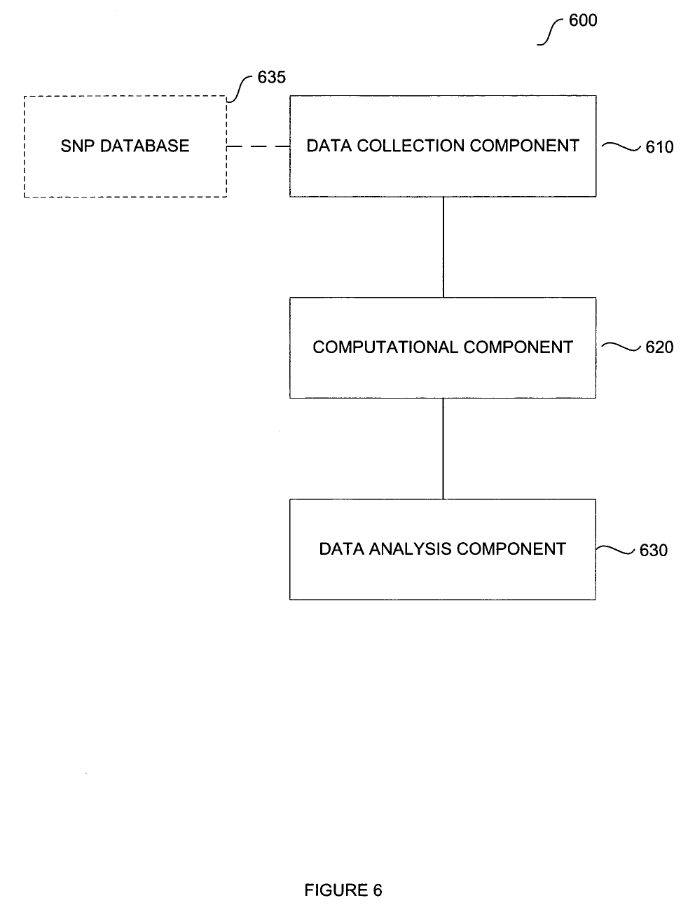

FIG. 6 illustrates a block diagram of a system 600 for conducting SNP analysis according to the present teachings. In one aspect, the system 600 comprises components/modules including; a data collection component 610, a computational component 620, and a data analysis component 630. These components 610, 620, 630 may be implemented separately or representative of a combined functionality of a singular multifunction component or module.

In accordance with the methods described above the data collection component 610 may be configured to provide functionality for selecting a data collection comprising information describing a plurality of single nucleotide polymorphisms (SNPs). This information may be obtained from a database or datastore 635 containing SNP information and associated characteristics and parameters to be used in the analysis. Alternatively, this information may be generated be typing a plurality of SNPs designated to be used during the analysis.

The computational component 620 provides functionality for determining informativeness for selected SNPs contained within the data collection based at least in part upon an identification of at least one region wherein allelic states for two or more SNPs of the data collection are correlated and wherein the informativeness for a selected SNP is reflected by the predictive value of the selected SNP in identifying allelic states for other SNPs within the region as described above.

Finally, the data analysis component 630 provides functionality for evaluating the informativeness of the SNPs within the data collection to identify a SNP subset that has a selected degree of predictive quality in predicting other SNPs in accordance with the methods and approaches described herein.

The following discussion exemplifies the effectiveness of the aforementioned approaches to SNP selection using various sample datasets. It will be appreciated that these results are merely illustrative and not meant to reflect any limitations of accuracy or utility of the present teachings. The sample datasets selected for this analysis comprise: a first dataset reflecting a chromosome-wide dataset from human chromosome 21 comprising approximately 24,047 SNPs typed on 20 haploid copies of the chromosomes; a second dataset derived from 71 individuals typed at 88 polymorphic sites in the Human lipoprotein lipase (LPL) gene; and a third dataset comprising 4102 SNPs distributed along most of the genomic span of chromosome 22 with a median spacing of 4 kb, genotyped by the 5′ nuclease assay (e.g. TagMan® Applied Biosystems, Foster City, Calif.) on 45 DNA samples of Caucasian individuals obtained from the NIGMS Human Variation Panel. In one aspect, the SNP density and sample size of this dataset is generally similar to that used by the International HapMap Project.

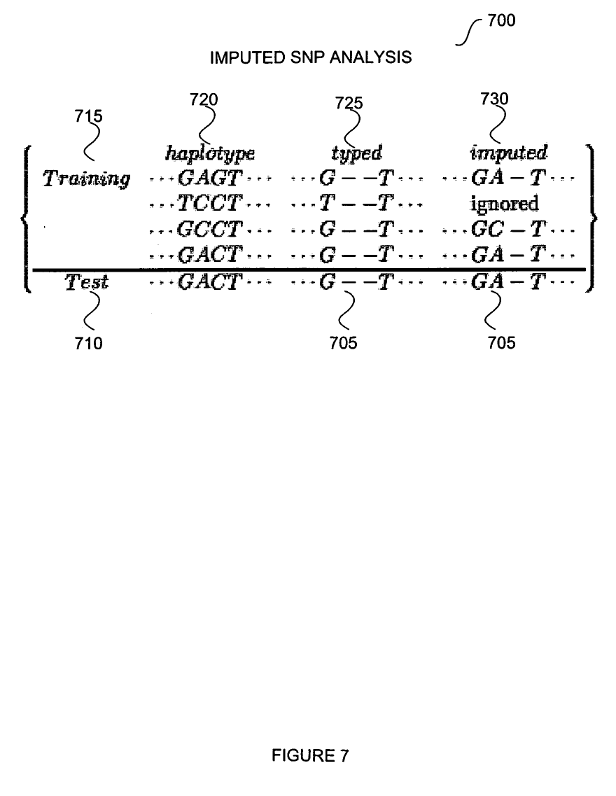

In evaluating possible solutions provided by the SNP selection methods leave-one-out cross validation was performed. For each haplotype in the dataset, the SNP selection method was trained on the rest of the dataset to determine a minimum informative SNP set. The performance of the SNP selection for the haplotype left out was evaluated by counting the number of alleles in that haplotype that were correctly imputed from the SNPs that were typed. As shown in table 700 of FIG. 7, the second SNP shown 705 in the test haplotype 710 may be imputed from the four training haplotypes 715. The “haplotype” column 720 illustrates the original haplotypes. The “typed” column 725 illustrates SNPs that are typed, the first and the fourth SNP. The “imputed” column 730 illustrates those haplotypes that may be used in the imputation of the second SNP 705. In one aspect, those haplotypes that are identical to the test haplotype on the first and fourth SNP are used in the imputation. As shown by way of example the ‘A’ may be imputed as of those haplotypes that agreed with the test haplotype on the first and fourth SNP, two haplotypes have an ‘A’ and one has a ‘C’.

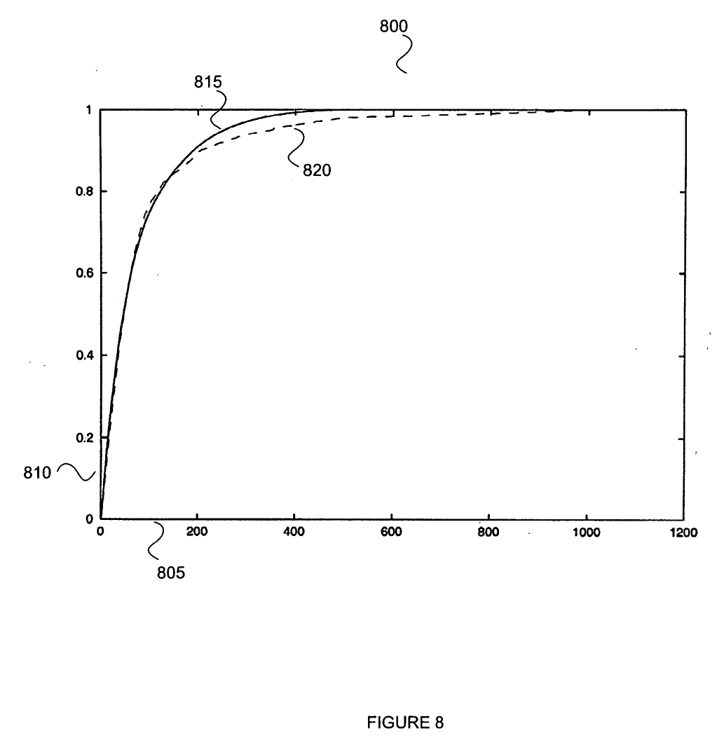

FIG. 8 is a graph 800 showing how the definition of informativeness described herein correlates with a practical benchmark of the value of an SNP subset illustrating its utility in predicting missing values. In this graph 800 informativeness and the number of SNPs correctly imputed in the leave-one-out cross-validation test are plotted as a function of the number of SNPs typed. The x-axis 805 shows the number of SNPs typed, and the y-axis 810 shows the fraction of total informativeness or total number of SNPs correctly imputed in a leave-one-out experiment. The solid (upper) curve 815 represents informativeness and the dashed (lower) curve 820 shows the fraction of SNPs that are correctly imputed in a leave-one-out experiment. The data set used is represented by the first 1000 SNPs of the Chromosome 21 data set using the no-four-gamete violation definition of blocks and neighborhoods. In this demonstration neighborhoods are restricted to have sizes of approximately no larger than 13.

As shown, informativeness and fraction of SNPs imputed in cross-validation studies closely track one another (e.g. see through approximately 80% of their maximal value achieved by both measures). The informativeness measure thus appears to be minimally affected by overfitting on sparse data over the observed range. For higher numbers of SNPs, cross-validated imputation fraction slightly lags informativeness. This latter observation may be consistent with the idea that a small fraction of the SNPs capture the common haplotype variants that account for most population variation and that are easily inferred from even very small population samples, but that a minority of the variation may be explained by rarer haplotype patterns that are more difficult to infer accurately from small population samples. Nonetheless, by way or example if only 20% of the SNPs are typed, it is possible to correctly impute approximately 90% of them using the methods described by the present teachings. Furthermore it would be expected that informativeness may track imputation accuracy even more closely when larger population samples are used for inference.

In one aspect, the accumulated accuracy over all haplotypes provides a global measure of the accuracy for the selected data set. Implicitly, all SNPs that are typed in the test haplotype may be considered to be correctly imputed. SNPs that are not typed may be imputed by looking at the typed SNPs in its neighborhood; if there are training haplotypes which have the same allele call on all the typed SNPs in their neighborhoods, then the allele may be determined by a majority vote of those haplotypes. If no such haplotype exists then the majority vote may be taken over all training haplotypes that have the same allele call on all but one of the typed SNPs in the neighborhood. Furthermore, if no haplotype exists having the same allele call on all but k SNPs typed in the neighborhood, then the allele call may be determined over all training haplotypes with the same allele call on all but k+1 SNPs typed. If the majority vote is determined to be ambiguous then the SNP may be counted as being predicted

The present teachings describe and illustrate novel mechanisms by which to identify and assess subsets of SNPs that are predictive of other SNPs within population samples. These approaches desirably avoid many of the difficulties conventional methodologies have experienced when attempting to identify appropriate SNPs for detailed haplotyping analysis, particularly when dealing with small population samples. In one aspect, the disclosed methods provide means for efficiently solving the problem of optimally finding minimum informative SNP subsets in many practical cases. These methods further be solved without having to resort to a haplotype block decomposition, which may be an important advantage given the uncertainty about the definition and utility of haplotype blocks. Additionally the disclosed methods overcome limitations of conventional methods that are restricted to those blocks and overcome problems such as not being able to tag SNPs outside the block limits and ignoring block to block LD. It is observed that there is considerable value to the disclosed methods for block-independent selection of informative SNP subsets, compared to both random selection and block-based selection techniques.

In one exemplary approach for defining neighborhoods of SNPs that can predict the state of another, one may take advantage of the uncertainty in the heuristics for finding haplotype blocks, and used as a neighborhood the union of alternative block decompositions that are possible for a given region. An alternative approach may be to utilize map distance thresholds as compared to a physical map to provide a block-free method that factors in map location in a meaningful way. The methods may also be used with informativeness measures or with other quality metrics more appropriate for a particular study design. For example, when there is more knowledge on the disease mode of inheritance or on the range of disease allele frequency, alternate objective functions may be considered such as maximizing the power for detecting association.

The present teachings therefore present a novel measure for the identification of subsets of SNPs that are predictive of other SNPs identified in population samples. One desirable feature of this measure is that it may avoid difficulties traditional linkage disequilibrium measures have experienced when applied to SNP selection, particularly when dealing with small population samples. Further presented is an approach for efficiently solving the problem of finding optimal or improved informative SNP subsets that may represent minimal compositions in certain cases. By not relying on haplotype block structures, the present teachings overcome the limitations of tagging methods that are restricted to those blocks and cannot tag SNPs outside and that ignore block-to-block LD. The present teachings are of considerable value for block-independent selection of informative SNP subsets, compared with both random selection and a leading block-based selection method. In certain embodiments, the aforementioned methods may not be significantly impaired by overfitting even when inferring from small population samples.

In one exemplary method for defining sensible neighborhoods of SNPs that can predict the state of another, one may take advantage of the uncertainty in the heuristics for finding haplotype blocks, and use as neighborhood the union of alternative block decompositions that are possible for a given region. Another alternative may be to use map distance thresholds as compared to a physical map. Such an approach may provide a block-free method that factors in map location in a meaningful way. A further advantage of the methods is that they may be used in connection with the informativeness measure described herein or with other desired quality metrics. For example, when there is knowledge of disease mode of inheritance or on the range of disease allele frequency, alternate objective functions may be considered such as maximizing the power for detecting association.

The various methods and techniques described above provide a number of examples of how the present teachings may be implemented and the potential benefits realized when applying them. It is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

It will be appreciated that while the present teachings are described in connection with SNP analysis, these methods may be readily adapted to the analysis and selection of other genetic markers and polymorphisms. For example, the disclosed methods may be configured for use in connection with microsatellite analysis, insertion/deletion analysis, biallelic polymorphism analysis, multiple nucleotide polymorphism analysis, and others as will be appreciated by one of skill in the art. As such the disclosed methods are conceived to be operable in these and other contexts without departing from the scope of the present teachings.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

All references cited in this specification are hereby incorporated by reference. The discussion of the references herein is intended merely to summarize the assertions made by their authors and no admission is made that any references constitutes prior art relevant to patentability.

What is claimed is:

1. A method for analyzing nucleotide sequence information, the method comprising:
selecting a data collection comprising information describing a plurality of genetic markers;
determining informativeness for selected genetic markers within the data collection based at least in part upon an identification of at least one neighborhood wherein allelic states for two or more genetic markers of the data collection are correlated and wherein the informativeness for a selected genetic marker is reflected by the predictive value of the selected genetic marker in identifying allelic states for other genetic markers within the region; and
evaluating the informativeness of the genetic markers within the data collection to identify a genetic marker subset that possesses a selected degree of predictive quality in predicting other genetic markers.

2. The method of claim 1 wherein, the selected degree of predictive quality of the genetic market subset is determined as a function of a reduced size of the genetic marker subset relative to the data collection from which it is derived.

3. The method of claim 2 wherein, the reduced size of genetic marker subset provides for improved computational performance in downstream applications over the data collection while retaining similar informational content.

4. The method of claim 2 wherein, the reduced size of genetic marker subset provides for reduced genotyping requirements over the data collection while retaining similar informational content.

5. The method of claim 1 wherein, the reduced size of genetic marker subset is determined as a function of the accuracy of predicting other alleles that have not been genotyped relative to the data collection from which it is derived.

6. The method of claim 1 wherein, the at least one neighborhood is determined on factors selected from the group consisting of: regions of linkage disequilibrium, linkage disequilibrium maps, chromosomal distances, haplotype blocks, and physical distances.

7. The method of claim 1 wherein, the genetic marker subset is predictive of both genetic markers within the data collection and also other genetic markers outside of the data collection.

8. The method of claim 7 wherein, the genetic marker subset is predictive of untyped genetic markers outside of the data collection.

9. The method of claim 1 wherein, the predictive quality of the genetic markers subset substantially preserves observed haplotype diversity within the data collection.

10. The method of claim 1 wherein, the informativeness of the genetic marker subset is evaluated upon the basis of factors selected from the group consisting of: haplotype correlations, genotype correlations, sequence correlations, allelic correlations, size correlations, distance correlations, linkage mapping correlations, linkage disequilibrium correlations, and linkage disequilibrium map correlations.

11. The method of claim 1 wherein, one or more genetic marker subsets are identified at a substantially genome-wide scale to facilitate disease association studies.

12. The method of claim 12 wherein, disease association studies are facilitated by reducing the number of genetic markers to be genotyped as compared to using the data collection.

13. The method of claim 1 wherein one or more genetic marker subsets are identified at a substantially genome-wide scale to facilitate drug response analysis and efficacy determination.

14. The method of claim 1 wherein the identified genetic marker subsets provide the ability to analyze genome-wide haplotypes without exhaustive genotyping of an entire genome.
The method of claim 1 wherein, the informativeness of each selected genetic marker is determined at least in part by evaluating which genetic markers each selected genetic marker has the most predictive value in identifying the allelic states for.

15. The method of claim 1 wherein, the identification of the genetic marker subset further comprises performing a data reduction operation to identify substantially the most informative genetic markers within the data collection.

16. The method of claim 1 wherein, the identification of the genetic markers subset further comprises performing a data reduction operation that evaluates combinations of informative genetic markers reducing overall size of the genetic marker subset while preserving a selected degree of predictive value in identifying allelic states for other genetic markers.

17. The method of claim 1 wherein, the genetic markers are selected from the group consisting of: single nucleotide polymorphisms (SNPs), microsatellites, insertions, deletions, biallelic polymorphisms, and multiple nucleotide polymorphisms.

18. A method for analyzing nucleotide sequence information, the method comprising:
selecting a data collection comprising genetic marker information describing a plurality of genetic markers;
determining at least one neighborhood comprising a plurality of genetic markers associated with the data collection;
identifying genetic markers associated with the at least one neighborhood having correlated allelic states;
determining at least one set of genetic markers selected from those genetic markers associated with the at least one neighborhood that infer one another; and
associating with the at least one set of genetic markers, a quality measure that reflects how well the one set of genetic markers can be used to characterize other genetic markers of the data collection.

19. The method of claim 18 wherein, the predictive quality of the genetic marker set is evaluated upon the basis of factors selected from the group consisting of: haplotype correlations, genotype correlations, sequence correlations, allelic correlations, size correlations, distance correlations, linkage mapping correlations, linkage disequilibrium correlations, and linkage disequilibrium map correlations.

20. The method of claim 18 further comprising performing a data reduction operation in which the quality measure associated each genetic marker set is evaluated to determine a genetic marker subset that possesses superior predictive quality in characterizing other genetic markers.

21. The method of claim 20 wherein, the determination of the genetic marker set further comprises evaluating combinations of genetic markers associated with the at least one neighborhood to reduce genetic marker subset size while preserving a selected degree of quality in characterizing other genetic markers.

22. The method of claim 20 wherein, the genetic markers of the genetic marker set possess a selected degree of predictive quality in characterizing other genetic markers contained within the data collection.

23. The method of claim 20 wherein, the genetic markers of the genetic marker set are used to characterize other genetic markers outside of the data collection.

24. The method of claim 18 wherein, the genetic markers are selected from the group consisting of: single nucleotide polymorphisms (SNPs), microsatellites, insertions, deletions, biallelic polymorphisms, and multiple nucleotide polymorphisms.

25. A system for analyzing nucleotide sequence information, the system comprising:
a data collection component that provides functionality for selecting a data collection comprising information describing a plurality of genetic markers;
a computational component that provides functionality for determining informativeness for selected genetic markers within the data collection based at least in part upon an identification of at least one region wherein allelic states for two or more genetic markers of the data collection are correlated and wherein the informativeness for a selected genetic marker is reflected by the predictive value of the selected genetic marker in identifying allelic states for other genetic markers within the region; and
a data analysis component that provides functionality for evaluating the informativeness of the genetic markers within the data collection to identify a genetic marker subset that has a selected degree of predictive quality in predicting other genetic markers.

26. The system of claim 25, wherein the computational component determines the informativeness of each selected genetic marker at least in part by evaluating which genetic markers each selected genetic marker substantially has the most predictive value in identifying the allelic states for.

27. The system of claim 25 wherein, the data analysis component further provides functionality for the identification of the genetic marker subset by performing a data reduction operation to identify substantially the most informative genetic markers within the data collection.

28. The system of claim 25 wherein, the computational component further provides functionality for identification of the genetic marker subset by performing a data reduction operation that evaluates combinations of informative genetic markers reducing overall size of the genetic marker subset while preserving the selected degree of predictive value in identifying allelic states for other genetic markers.

29. The method of claim 25 wherein, the predictive quality of the genetic marker subset is evaluated upon the basis of factors selected from the group consisting of: haplotype correlations, genotype correlations, sequence correlations, allelic correlations, size correlations, distance correlations, linkage mapping correlations, linkage disequilibrium correlations, and linkage disequilibrium map correlations.

30. The system of claim 25 wherein, the genetic markers are selected from the group consisting of: single nucleotide polymorphisms (SNPs), microsatellites, insertions, deletions, biallelic polymorphisms, and multiple nucleotide polymorphisms.

31. An apparatus comprising a computer readable medium having instructions stored thereon to analyze nucleotide sequence information by the steps of:
selecting a data collection comprising genetic marker information describing a plurality of genetic markers;
determining at least one neighborhood associated with the data collection;
identifying genetic markers associated with the at least one neighborhood;
determining at least one set of genetic markers selected from those genetic markers associated with the at least one neighborhood that infer one another; and
associating with the at least one at least one set of genetic markers, a quality measure that reflects how well the one set of genetic markers can be used to characterize other genetic markers.

32. The method of claim 31 wherein, the genetic markers are selected from the group consisting of: single nucleotide polymorphisms (SNPs), microsatellites, insertions, deletions, biallelic polymorphisms, and multiple nucleotide polymorphisms.

33. The method of claim 31 wherein, the at least one neighborhood is determined on factors selected from the group consisting of: regions of linkage disequilibrium, linkage disequilibrium maps, chromosomal distances, haplotype blocks, and physical distances.
